(19) United States
(12) Patent Application Publication
(10) Pub. No.: US 2006/0073211 A1
(43) Pub. Date: Apr. 6, 2006

(54) Cellulite formulation containing whole egg

(76) Inventors: Michael Marenick, Glen Ridge, NJ (US); Robert L. Albright, Southampton, PA (US)

Correspondence Address: Dan M. De La Rosa, Esq., Suite 27H, 345 E 80th St., New York, NY, 10021, US

(21) Appl. No.: 11/294,838

(22) Filed: Dec. 7, 2005

Related U.S. Application Data
(63) Continuation-in-part of application No. 10/260,344, filed on Oct. 1, 2002.
Application No. 10/260,344 is a continuation of application No. 10/039,793, filed on Jan. 8, 2002, now abandoned.
(60) Provisional application No. 60/298,874, filed on Jun. 18, 2001.

(51) Int. Cl. A61K 35/34; A61K 36/15; A61K 36/16; A61K 36/185; A61K 36/23; A61K 36/258; A61K 36/282; A61K 36/288; A61K 36/48; A61K 36/899; A61K 36/84

(52) U.S. Cl. 424/581; 424/725; 424/757; 424/764; 424/728; 424/745; 424/762; 424/750; 424/726; 424/737; 424/752; 424/733; 424/767

(57) ABSTRACT

A skin care formulation is provided, the formulation comprises whole egg, at least one herbal extract, at least one humectant and at least one aromatherapeutical substance or essential oil, whereby the formulation is used to reduce the visible appearance of cellulite. The formulation contains no caffeine, no aminophylline and no caffeine derivatives.

### RELATED APPLICATION

This application is a continuation-in-part of U.S. application Ser. No. 10/260,344, entitled “SKIN CARE PRODUCTS CONTAINING WHOLE EGG”, which was filed on Oct. 1, 2002, which has now been given a notice of allowance and is a continuation of U.S. application Ser. No. 10/039,793, entitled HYDROLYZED WHOLE EGG PRODUCTS & RELATED METHODS”, which was filed on Jan. 8, 2002 and is now abandoned and is related to Provisional U.S. Application Ser. No. 60/298,874, entitled “A SKIN CARE PRODUCT AND A METHOD OF PRODUCING SAME” which was filed on Jun. 18, 2001.

### BACKGROUND OF THE INVENTION

1. Field of the Invention

This invention relates to a skin care product containing whole eggs wherein the eggs are utilized as a carrier of the active ingredients in the product and is used to achieve the desired effects of the product. More specifically, the present invention relates to a whole egg product for use as a cosmetic, pharmaceutical and/or medicinal product applicable for soothing sore muscles, expediting the healing and repairing process of the skin and reducing the visual appearance of cellulite.

2. Description of the Related Art

There are numerous cosmetic and skin care products that use components of eggs as major ingredients in their formulation. For example, some cosmetic or skin care products use egg whites in their formulation. Other cosmetic and skin care products use extracts of yokes in their formulation. The use of whole egg has never been achieved since the mixing and/or shearing process of the manufacturing of the product will cause the egg components to coagulate and form mayonnaise. There have also been problems with incorporating the whole egg in the formations since the shearing and heating process of most processes have denatured the proteins in the eggs and have inactivated the active ingredients in the egg which provides for its excellent carrier/delivery properties. In addition, there are no cosmetic, medicinal, pharmaceutical and/or cosmopharmaceutical product that utilize whole eggs as carriers for their active ingredients.

### SUMMARY OF THE INVENTION

In one embodiment, the present invention provides for a cellulite formulation comprising whole egg, at least one herbal extract, at least one humectant substance and at least one aromatherapeutical substance, whereby the formulation is used to reduce the visible appearance of cellulite. In another embodiment, the formulation contains no caffeine, no aminophylline and no caffeine derivatives. In still another embodiment, the herbal extract in the formulation is selected from a group consisting of cumin extract, elder berry extract, caraway extract, sage extract, parsley extract, cowslip extract, grapefruit seed extract and mixtures thereof. In yet another embodiment, the humectant substance is selected from a group consisting of glycerin, glycols, butylene glycol, propylene glycol, pentylene glycol and mixtures thereof. In still yet another embodiment, the aromatherapeutical substance comprises essential oils and essential oils are selected from a group consisting of lavender oil, geranium oil, juniper berry oil, bay leaf oil and mixtures thereof.

In a further embodiment, the herbal extract can also be selected from a group consisting of angelica extract, ashwaganda extract, astragalus extract, bilberry extract, black cohosh extract, blue cohosh extract, blessed thistle extract, burdock extract, California poppy extract, cactus extract, celandine extract, chamomile extract, chaparral extract, chaste tree extract, chickweed extract, cleavers extract, coltsfoot extract, corn silk extract, cramp bark extract, damiana extract, dandelion extract, devil's claw extract, dong quai extract, echinacea extract, sib ginseng extract, eyebright extract, false unicorn root extract, fenugreek extract, ginkgo extract, ginseng root extract, goldenrod extract, goldenseal extract, gotu kola extract, horsetail extract, hyssop extract, kava kava extract, lady's mantle extract, licorice root extract, milk thistle extract, motherwort extract, nettle extract, oats extract, Oregon grape root extract, osha root extract, passionflower extract, pau d'arco extract, plantain extract, red clover extract, red root extract, reishi mushroom extract, sarsaparilla extract, saw palmetto extract, St. John's wort extract, schisandra berry extract, shepherds purse extract, skullcap extract, slippery elm extract, usnea lichen extract, uva ursi extract, valerian extract, wild cherry extract, wild yam extract, wormwood extract, yellow dock extract, yucca root extract and mixtures thereof In a further embodiment, the herbal extract of the present invention also uses botanical extracts.

In still a further embodiment, the aromatherapeutical substance comprises essential oils and the essential oils can be selected from a group consisting of absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemon oil, lemongrass oil, lemon eucalyptus oil, lemon myrtle oil, lemon 5 fold oil, lican oil, lime oil, litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palnarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balaam oil, turmeric oil, valerian root oil, vanilla 10 fold oil, vetivewr oil, wintergreen oil, wild verbena oil, wormwood oil, yarrow oil, ylang ylang oil and mixtures thereof.

In still yet a further embodiment, the formulation further comprises at least one emollient substance, and the emollient substance is selected from a group consisting of sweet almond oil, Squalene, apricot oil, safflower oil, sunflower oil and mixtures thereof. In another further embodiment, the whole egg may be selected from a group consisting of poultry eggs, reptile eggs and mixtures thereof, and the poultry eggs being selected from a group consisting of chicken eggs, duck eggs, emu eggs, geese eggs, guinea fowl eggs, ostrich eggs, partridge eggs, peafowl eggs, pheasant eggs, pigeon eggs, rhea eggs, swan eggs, turkey eggs and mixtures thereof

In still another further embodiment, the formulation has a shelf life of at least one year. In yet another further embodiment, the formulation can have a shelf life of two years and more. In still yet another further embodiment, the whole egg comprises dehydrated whole egg, and in another embodiment, the dehydrated whole egg is whole egg powder. In another embodiment, the dehydrated egg is hydrated during the manufacturing process of said formulation.

In still another embodiment, the present invention also provides for an application of use for treating cellulite, and the application comprises whole egg, at least one essential oil, at least one humectant substance and at least one herbal extract. In yet another embodiment, the essential oil is selected from a group consisting of lavender oil, geranium oil, juniper berry oil, bay leaf oil and mixtures thereof. In still yet another embodiment, the humectant substance is selected from a group consisting of glycerin, glycols, butylene glycol, propylene glycol, pentylene glycol and mixtures thereof In a further embodiment, the herbal extract is selected from a group consisting of cumin extract, elder berry extract, caraway extract, sage extract, parsley extract, cowslip extract, grapefruit seed extract and mixtures thereof. In yet a further embodiment, the application of use further comprises at least one emollient substance, and the emollient substance is selected from a group consisting of sweet almond oil, Squalene, apricot oil, safflower oil, sunflower oil and mixtures thereof.

In still a further embodiment, the present invention provides for a method of manufacturing a cellulite product, and the method comprises admixing whole egg, at least one herbal extract, at least one humectant substance and at least one aromatherapeutical substance; the aromatherapeutical substance comprising essential oils, and the formulation is used to reduce the visible appearance of cellulite, and contains no caffeine, no aminophylline and no caffeine derivatives.

In still yet further embodiment, the method further comprises admixing aloe barbadensis leaf juice and at least one emollient substance. In another further embodiment, the cellulite product manufactured by the above method has a shelf life of at least one year. In still another further embodiment, the whole egg used in the manufacturing method comprises dehydrated whole egg, and the dehydrated whole egg is whole egg powder. In yet another embodiment, the whole egg powder is hydrated during the manufacturing method of the cellulite product.

In another embodiment, the present invention provides for a skin care product and the product comprises dehydrated whole egg, at least one emollient substance and at least one humectant substance and the dehydrated whole egg is hydrated during the manufacturing process and the dehydrated whole egg may be selected from a group consisting of poultry eggs, reptile eggs and mixtures thereof, and the poultry eggs being selected from a group consisting of chicken eggs, duck eggs, emu eggs, geese eggs, guinea fowl eggs, ostrich eggs, partridge eggs, peafowl eggs, pheasant eggs, pigeon eggs, rhea eggs, swan eggs, turkey eggs and mixtures thereof

In yet another embodiment, the present invention also provides for a skin care formulation comprising whole egg powder, at least one emollient substance and at least one humectant substance and the formulation has a shelf life of at least one year and the whole egg powder may be selected from a group consisting of poultry eggs, reptile eggs and mixtures thereof, and the poultry eggs being selected from a group consisting of chicken eggs, duck eggs, emu eggs, geese eggs, guinea fowl eggs, ostrich eggs, partridge eggs, peafowl eggs, pheasant eggs, pigeon eggs, rhea eggs, swan eggs, turkey eggs and mixtures thereof

In still yet another embodiment, the present invention provides for a method of manufacturing a skin care formulation and the method comprises admixing dehydrated whole egg, at least one emollient substance and at least one humectant substance and the dehydrated whole egg is hydrated during the manufacturing process and the dehydrated whole egg may be selected from a group consisting of poultry eggs, reptile eggs and mixtures thereof, and the poultry eggs being selected from a group consisting of chicken eggs, duck eggs, emu eggs, geese eggs, guinea fowl eggs, ostrich eggs, partridge eggs, peafowl eggs, pheasant eggs, pigeon eggs, rhea eggs, swan eggs, turkey eggs and mixtures thereof.

For purposes of this invention, the term “egg” is defines as the whole egg, which consists of the yolk (yellow) and the albumin (white) of the egg. In another embodiment, the term “egg” is defines as the whole egg, which consists of the shell, the yolk (yellow) and the albumin (white) of the egg. For purposes of this invention, the term “dehydrated whole egg” is defined as whole egg, which has had the water extracted from it. In one embodiment, dehydrated whole egg is the same as whole egg powder. For purposes of this invention, the term “essential oil” is defined as “volatile oily substance produced by many plants” and essential oils contain vitamins, hormones, antibiotics and/or antiseptics. In addition, the term “herbal extract” is defined as an extract derived from a seed producing annual, biannual or herbaceous perennial plants.

In another embodiment, the present invention relates to a formulation comprising at least one hydrolyzed whole egg, at least one emollient substance and at least one humectant substance. The term “hydrolyzed whole egg” is defined as a whole egg which through a hydrolysis process of breaking down the larger molecules into smaller molecules of the egg.

For purposes of this invention, the term “emollient substance” is defined as any natural or synthetic oil and their compound equivalents. The term “humectant substance” for the purposes of this invention is defined as glycerin and/or any other chemical of natural or synthetic origin. In another embodiment, the humectant substance is selected from a group consisting of glycerin, butylene glycol, propylene glycol, pentylene glycol and mixtures thereof In yet another embodiment, the emollient substance is selected from a group consisting of Prunus Amygdalus Dulis (Sweet Almond) Oil, Squalene, Prunus Armeniaca (Apricot) Kernel Oil, Carthamus Tinetorius (Safflower) Oil, Helianthus Annuus (Sunflower) Seed Oil and extracts thereof and mixtures thereof In still another embodiment, the formulation can be utilized as a cosmetic, cosmopharmaceutical and pharmaceutical formulation.

In a further embodiment, the present invention relates to a formulation comprising: whole egg and lactoferrin and/or colostrum. In still a further embodiment, the present invention provides for a formulation comprising: whole egg, at least one emollient substance, at least one humectant substance, and lactoferrin and/or colostrum. In still another embodiment, the colostrum is bovine colostrum.

In yet a further embodiment, the present invention relates to a formulation comprising whole egg and methyl sulfonyl methane (MSM). In still yet a further embodiment, the formulation comprises whole egg, at least one emollient substance, at least one humectant substance, and methyl sulfonyl methane (MSM). In a further embodiment, the present invention provides a formulation comprising whole egg, methyl sulfonyl methane (MSM) and lactoferrin and/or colostrum. In another further embodiment, the formulation comprises: whole egg, at least one emollient substance, at least one humectant substance, methyl sulfonyl methane (MSM) and lactoferrin and/or colostrum. In still another embodiment, the colostrum is bovine colostrum.

In still yet another formulation, the present invention relates to a cellulite formulation comprising at least one whole egg, at least one emollient substance, at least one humectant substance and at least one aromatherapeutical substance. In a further embodiment, the humectant substance is selected from a group consisting of glycerin, butylene glycol, propylene glycol, pentylene glycol and mixtures thereof In yet a further embodiment, the emollient substance is selected from a group consisting of Prunus Amygdalus Dulis (Sweet Almond) Oil, Squalene, Prunus Armeniaca (Apricot) Kernel Oil, Carthamus Tinetorius (Safflower) Oil, Helianthus Annuus (Sunflower) Seed Oil and extracts thereof and mixtures thereof.

In still yet a further embodiment, the aromatherapeutical substance is selected from a group consisting of Lavendula Angustifolia (Lavender) oil, Geranium Maculatum Oil, Citrus Grandis (Grapefruit) oil, Juniperus Communis Oil, Pimenta Acris (Bay) Oil, Lavendula Hybrida, Geranium Robertianum, Geranium Thunbergil, Citrus Aurantium Dulsis (Orange) Oil, Citrus Nobilis (Mandarin Orange) Oil, Citrus Limonum (Lemon) Oil and extracts thereof and mixtures thereof For purposes of this invention, the term “aromatherapeutical substance” is defined as a combination of pure essential oils with aromatic properties.

In another further embodiment, the present invention relates to skin care formulation comprising at least one whole egg, at least one emollient substance, at least one humectant substance and at least one skin nourishing/wound healing substance. In still another further embodiment, the humectant substance is selected from a group consisting of glycerin, butylene glycol, propylene glycol, pentylene glycol and mixtures thereof. In yet another further embodiment, the emollient substance is selected from a group consisting of Prunus Amygdalus Dulis (Sweet Almond) Oil, Squalene, Prunus Armeniaca (Apricot) Kernel Oil, Carthamus Tinetorius (Safflower) Oil, Helianthus Annuus (Sunflower) Seed Oil and extracts thereof and mixtures thereof.

In still yet another further embodiment, the skin nourishing/wound healing substance is selected from a group consisting of aloe barbadensis leaf juice, white willow bark, and extracts thereof and mixtures thereof The term “skin nourishing/wound healing substance” is defined as any material that will assist in or promote the skin healing and/or normalizing process. In another embodiment, the skin care formulation further comprises an acne drug and the formulation functions as an acne formulation. In still another embodiment, the active drug is salicylic acid.

In a further embodiment, the present invention also relates to a muscle soothing formulation comprising at least one whole egg, at least one emollient substance, at least one humectant substance and a muscle soothing substance. In still a further embodiment, the humectant substance is selected from a group consisting of glycerin, butylene glycol, propylene glycol, pentylene glycol and mixtures thereof In yet a further embodiment, the emollient substance is selected from a group consisting of Prunus Amygdalus Dulis (Sweet Almond) Oil, Squalene, Prunus Armeniaca (Apricot) Kernel Oil, Carthamus Tinetorius (Safflower) Oil, Heilanthus Annuus (Sunflower) Seed Oil and extracts thereof and mixtures thereof

In still yet a further embodiment, the muscle soothing substance comprises a blend of Menthol, Methyl Salicylate, Eucalyptus Globulus Oil, Camphor, and Mentha Piperita (Peppermint) Oil. For purposes of this invention, the “muscle soothing substance” is defined as any active ingredient or pure substance that can penetrate and cause sensation to the muscle tissue.

In another further embodiment, the present invention relates to whole egg formulations manufactured by a method comprising: preparing an aqueous phase by mixing water, triethanolamine, glycerin and methyl paraben and heating the aqueous phase to a temperature from about 70 degrees Celsius to about 80 degrees Celsius; preparing a separate oil phase by mixing sweet almond oil, stearic acid, glyceryl stearate, and propylparaben and heating the oil phase to a temperature from about 90 degrees Celsius to about 100 degrees Celsius; adding whole egg (dehydrated whole egg or whole egg powder) to the oil phase while stopping the heating process and adding the oil phase to the aqueous phase with agitation to form an emulsion; and upon formation of the emulsion, cooling the entire formulation.

In still another further embodiment, the method further comprising adding additional substances to said formulation during said cooling process. In yet another further embodiment, the formulation made by the above method may be utilized as a cosmetic, cosmopharmaceutical and pharmaceutical formulation depending on what additional substance(s) are added to the formulation during the cooling process.

In another embodiment, the present invention relates to a skin care formulation manufactured by a process comprising: preparing an aqueous phase by mixing water, triethanolamine, trisodium EDTA, glycerin and methyl paraben and heating the aqueous phase to a temperature from about 70 degrees Celsius to about 80 degrees Celsius; preparing a separate oil phase by mixing cetearyl alcohol, sweet almond oil, cetyl alcohol, stearic acid, glyceryl stearate, sorbitan stearate, tocopherol, retinyl palmitate, tetrahexyldecyl ascorbate and propylparaben and heating the oil phase to a temperature from about 90 degrees Celsius to about 100 degrees Celsius; adding whole egg to the oil phase while stopping the heating process and adding the oil phase to the aqueous phase with agitation to form an emulsion; upon formation of the emulsion, cooling the entire formulation; and adding glycerin, salicylic acid and phenoxyethanol and shearing the entire formulation. In still another embodiment, the formulation made by the above process may be used for acne skin care. Any formulation or product that contains whole egg or has whole egg incorporated into its formulation, has used the above-mentioned process or related process. In one embodiment, the ideal temperature for admixing the whole egg to the oil is from about 60 to about 90 degrees Celsius.

In yet another embodiment, the present invention further relates to a muscle soothing formulation manufactured by a process comprising: preparing an aqueous phase by mixing water, triethanolamine, glycerin, trisodium and methyl paraben and heating the aqueous phase to a temperature from about 70 degrees Celsius to about 80 degrees Celsius; preparing a separate oil phase by mixing sweet almond oil, cetearyl alcohol, stearic acid, glyceryl stearate, cetyl lactate, tocopherol, retiryl palmitate, tetrahexyldecyl ascorbate and propylparaben and heating the oil phase to a temperature from about 90 degrees Celsius to about 100 degrees Celsius; adding whole egg to the oil phase while stopping the heating process and adding the oil phase to the aqueous phase with agitation to form an emulsion; upon formation of the emulsion, cooling the entire formulation; and adding cyclomethicone, menthol, methyl salicylate, eucalyptus globules oil, camphor, peppermint oil, phenoxyethanol and chlorophyll and shearing the entire formulation.

In a further embodiment, the present invention relates to a cellulite formulation manufactured by a process comprising: preparing an aqueous phase by mixing water, triethanolainie, glycerin, propylene glycol and methyl paraben and heating the aqueous phase to a temperature from about 70 degrees Celsius to about 80 degrees Celsius; preparing a separate oil phase by mixing sweet almond oil, cetyl lactate, stearic acid, paraffin, sorbitan stearate, glyceryl stearate, cyclomethicone and dimethicone copotyol, and propylparaben and heating the oil phase to a temperature from about 90 degrees Celsius to about 100 degrees Celsius; adding whole egg to the oil phase while stopping the heating process and adding the oil phase to the aqueous phase with agitation to form an emulsion; upon formation of the emulsion, cooling the entire formulation; and adding grapefruit oil, lavender oil, geranium maculatum oil, juniperus communis oil, cumen extract, sambucus nigra extract, caraway extract, sage extract, parsley extract, primula veris extract and phenoxyethanol and shearing the entire formulation.

In still a further embodiment, the present invention relates to a method of manufacturing a formulation, the method comprising: shearing at least one emollient substance and at least one humectant substance; adding at least one whole egg to the oil phase of the mixture and then adding the oil phase to the aqueous phase to form an emulsion; and upon formation of the emulsion, cooling the formulation. In yet a further embodiment, the humectant substance is selected from a group consisting of glycerin, butylene glycol, propylene glycol, pentylene glycol and mixtures thereof In yet another further embodiment, the emollient substance is selected from a group consisting of Prunus Amygdalus Dulis (Sweet Almond) Oil, Squalene, Prunus Armeniaca (Apricot) Kernel Oil, Carthamus Tinetorius (Safflower) Oil, Helianthus Annuus (Sunflower) Seed Oil and extracts thereof and mixtures thereof. In still yet a further embodiment, the formulation may be used as a cosmetic, cosmopharmaceutical and pharmaceutical formulation depending on what additional substance(s) are added to the formulation.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessary to scale, some features may be exaggerated to show details of particular components. Therefore, specific structural and functional details disclosed herein are not to be interpreted as limiting, but merely as a basis for the claims and as a representative basis for teaching one skilled in the art to employ the present invention in various ways.

The present invention provides cosmetic, cosmopharmaceutical and pharmaceutical formulations containing whole egg(s). Whole eggs are a great source of nutrients and function as carriers/deliverers of various active and inactive ingredients to the respective human/animal bodily components, organs, tissues, cells, etc. The present invention utilizes chicken eggs but can use any and all types of eggs, including but not limited to, poultry and reptile eggs.

The formulation of the present invention includes, but is not limited to, a skin care formulation, a muscle soothing formulation and a cellulite formulation. There are three unique products, discussed in detail below, that have radically different functions ranging from soothing sore muscles, to expediting the repairing process of the skin and reducing the visual appearance of cellulite. The common factor that is shared by all of these products is the use of whole eggs to achieve the desired effects in each case and the unique processing method of incorporating the egg within the formulation and allowing the whole egg to act as a carrier of the active ingredients in each case.

The specific examples below will enable the present invention to be better understood. However, they are given merely by way of guidance and do not imply any limitation to this invention.

### EXAMPLE 1

HEALFAST™ Skin Care Formulation

The skin care formulation of the present invention is designed to treat eczema, psoriasis, sunburns, razor burns, blisters, acne, insect bites, burns, cuts, bruises and dry skin. The skin care formulation of the present invention, may be manufactured by the following process: preparing an aqueous phase by mixing water, triethanolamine, trisodium EDTA, glycerin and methyl paraben and heating the aqueous phase to a temperature from about 70 degrees Celsius to about 80 degrees Celsius; preparing a separate oil phase by mixing cetearyl alcohol, sweet almond oil, cetyl alcohol, stearic acid, glyceryl stearate, sorbitan stearate, tocopherol, retinyl palmitate, tetrahexyldecyl ascorbate and propylparaben and heating the oil phase to a temperature from about 90 degrees Celsius to about 100 degrees Celsius; adding whole egg to the oil phase while stopping the heating process and adding the oil phase to the aqueous phase with agitation to form an emulsion; upon formation of the emulsion, cooling the entire formulation; and adding glycerin, salicylic acid and phenoxyethyanol and shearing the entire formulation. Table 1 below indicates the ingredients of the skin care product of the present invention including the sequence of addition into the formulation and the % W/W of each ingredient relate to the entire formulation. Sequence 1 below forms the aqueous phase and sequence 2 forms the oil phase. Sequence 3 and 4 are added to the emulsion.

TABLE 1
Skin Care Ingredients for HEALFAST ™ Formulation

| SEQUENCE | TRADENAME | INCI NAME | % W/W |
|---|---|---|---|
| 1 | Water | Water (Aqua) | 50-80 |
| 1 | Triethanola- | Triethanolamine | 0.05-5.00 |
| | mine 99% | | |
| 1 | Hampene Na3T | Trisodium EDTA | 0.10-1.00 |
| 1 | Glycerin | Glycerin | 0.10-3.00 |
| 1 | Methylparaben | Methylparaben | 0.10-5.00 |
| 2 | Lipowax D | Cetearyl Alcohol | 0.10-5.00 |
| | | (and) Ceteareth-20 | |
| 2 | Sweet Almond Oil | Sweet Almond | 0.10-10.00 |
| | | (Prunus Amygdalus | |
| | | Dulcis) Oil | |
| 2 | Lipocol | Cetyl Alcohol | 0.10-5.00 |
| 2 | Stearic Acid XXX | Stearic Acid | 0.10-5.00 |
| 2 | Lipomulse 165 | Glyceryl Stearate | 0.10-5.00 |
| | | (and) PEG-100 | |
| | | Stearate | |
| 2 | Sorbitan | Sorbitan Stearate | 0.10-5.00 |
| | Stearate | | |
| 2 | Covi-ax T-50 | Tocopherol | 0.01-1.00 |
| 2 | Vitamin A | Retinyl Palmitate | 0.10-3.00 |
| | Palmitate | | |
| 2 | BVOSC (Barnet) | Tetrahexyldecyl | 0.10-3.00 |
| | | Ascorbate | |
| 2 | Propylparaben | Propylparaben | 0.10-3.00 |
| 2 | Whole Egg | Whole Egg | 0.10-10.00 |
| 3 | Glycerin | Glycerin | 0.10-5.00 |
| 3 | Salicylic Acid | Salicylic Acid | 0.01-0.49 |
| 4 | Emmeressence | Phenoxyethanol | 0.10-3.00 |
| | 1160 Rose | | |

In another embodiment of the present invention, the above skin care formulation (HEALFAST™) may be embedded or incorporated onto a patch or bandage, which can be placed onto the target area of the human body (for example, a cut or laceration on the forearm). The patch or bandage may be of any shape or size and it comprises an adhesive strip for attaching the pad or bandage onto the human user and a pad containing the skin care formulation, which is placed directly upon the target area. The patch or bandage may further comprise removable tabs (which are removed at the moment of use) and a sterile wrapper or packaging.

The skin care formulation may be embedded or incorporated onto or into the pad through any and all techniques utilized in embedding medicament, ointment or other substances. The pad and adhesive strip may be constructed of any stretchable, bendable or breathable material, including various fabrics. The pad and adhesive strip may also be constructed of water resistant materials. The pad may be an absorbent cushion pad that has excellent sorbent properties. The patch or bandage may be dermatologically tested and hypoallergenic.

In still another embodiment, the muscle soothing formulation and cellulite formulation discussed below may also be embedded or incorporated onto patches or bandages. Like the HEALFAST™ patches or bandages, the muscle soothing and cellulite patches or bandages may also be attached onto the target area of the human anatomy (for example, the cellulite patch may be placed upon the woman's thighs or the muscle soothing patch may be placed upon an aching back or foot). The patches may also contain concentrated amount of the formulation and based upon the formulation, the patch or bandage may be designed to be used for hours or for days and even for weeks. The patches or bandages are utilized to apply the formulation directly onto target areas and may provide a less messy alternative to direct application of the formulation onto the user's body. The patches will also prevent contact of the formulation with the user's clothing.

### EXAMPLE 2

ULTRA SOOTHE® Muscle Soothing Formulation

The muscle soothing formulation of the present invention is designed to treat muscle aches and pains, and is applicable for all muscle parts and joints. The muscle soothing formulation of the present invention may be manufactured by the following method: preparing an aqueous phase by mixing water, triethanolainine, glycerin, trisodium and methyl paraben and heating the aqueous phase to a temperature from about 70 degrees Celsius to about 80 degrees Celsius; preparing a separate oil phase by mixing sweet almond oil, cetearyl alcohol, stearic acid, glyceryl stearate, cetyl lactate, tocopherol, retiryl palmitate, tetrahexyldecyl ascorbate and propylparaben and heating the entire mixture to a temperature from about 90 degrees Celsius to about 100 degrees Celsius; adding whole egg to the oil phase while stopping the heating process and adding the oil phase to the aqueous phase to form an emulsion; upon formation of the emulsion, cooling the entire formulation; and adding cyclomethicone, menthol, methyl salicylate, eucalyptus globules oil, camphor, peppermint oil, phenoxyethanol and chlorophyll and shearing the entire formulation. Table 2 below indicates the ingredients of the muscle soothing product of the present invention including the sequence of addition into the formulation and the % W/W of each ingredient relate to the entire formulation. Sequence 1 below forms the aqueous phase and sequence 2 forms the oil phase. Sequence 3, 4, 5, 6 and 7 are added to the emulsion.

TABLE 2
Muscle Soothing Ingredients for ULTRA
SOOTHE ® Formulation

| SEQUENCE | TRADENAME | INCI NAME | % W/W |
|---|---|---|---|
| 1 | Water | Water (Aqua) | 20-60 |
| 1 | Glycerin | Glycerin | 0.10-10.00 |
| 1 | Methylparaben | Methylparaben | 0.10-0.30 |
| 1 | Hampene Na3T | Trisodium EDTA | 0.10-0.30 |
| 1 | Triethanolamine | Triethanolamine | 0.01-3.00 |
| 2 | Stearic Acid | Stearic Acid | 0.50-5.00 |
| 2 | Lipowax D | Cetearyl Alcohol | 2.50-7.50 |
| | | (and) Ceteareth-20 | |
| 2 | Almond Oil | Sweet Almond | 5.0-15.00 |
| | | (Prunus Amygdalus | |
| | | Dulcis) Oil | |
| 2 | Lipomulse 165 | Glyceryl Stearate | 3.50 |
| | | (and) PEG-100 | |
| | | Stearate | |
| 2 | Ceraphyl 28 | Cetyl Lactate | 0.30-4.00 |
| 2 | Whole Egg | Whole Egg | 0.10-10.00 |
| 2 | Covi-ax T-50 | Tocopherol | 0.10-0.50 |
| 2 | Vitamin A | Retinyl Palmitate | 0.10-0.50 |
| | Palmitate | | |
| 2 | BVOSC (Barnet) | Tetrahexyldecyl | 0.10-1.00 |
| | | Ascorbate | |
| 2 | Propylparaben | Propylparaben | 0.10-0.30 |
| 3 | Sepigel 305 | Polyacrylamide | 2-5 |
| | | (and) C13-14 | |
| | | Isoparaffin | |
| | | (and) Laureth-7 | |
| 4 | Dow Corning | Cyclomethicone | 1.00-5.00 |
| | 345 Fluid | | |
| 5 | Menthol | Menthol | 2-10 |
| 5 | Methyl | Methyl Salcylate | 2-10 |
| | Salicylate | | |
| 5 | Eucalyptus Oil | Eucalyptus Globulus | 0.10-5.00 |
| | | Oil | |
| 5 | Camphor | Camphor | 2-10 |
| 5 | Peppermint Oil | Peppermint (Mentha | 0.10-5.00 |
| | | Piperita) Oil | |
| 6 | Emmeressence | Phenoxyethanol | 0.10-3.00 |
| | 1160 Rose | | |
| 7 | Chlorophyllin | Chlorophyll | q.s. |
| | Coper Complex | | |

### EXAMPLE 3

CELLUTONE® Cellulite Formulation

The cellulite formulation of the present invention is designed to reduce the visual appearance of cellulite. The cellulite formulation of the present invention may be manufactured by the following method: preparing an aqueous phase by mixing water, triethanolamine, glycerin, propylene glycol and methyl paraben and heating the aqueous phase to a temperature from about 70 degrees Celsius to about 80 degrees Celsius; preparing a separate oil phase by mixing sweet almond oil, cetyl lactate, stearic acid, paraffin, sorbitan stearate, glyceryl stearate, cyclomethicone and dimethicone copotyol, and propylparaben and heating the oil phase to a temperature from about 90 degrees Celsius to about 100 degrees Celsius; adding whole egg to the oil phase while stopping the heating process and adding the oil phase to the aqueous phase with agitation to form an emulsion; upon formation of the emulsion, cooling the entire formulation; and adding grapefruit oil, lavender oil, geranium maculatum oil, juniperus communis oil, cumen extract, sambucus nigra extract, caraway extract, sage extract, parsley extract, primula veris extract and phenoxyethanol and shearing the entire formulation. Table 3 below indicates the ingredients of the cellulite product of the present invention including the sequence of addition into the formulation and the % W/W of each ingredient relate to the entire formulation. Sequence 1 below forms the aqueous phase and sequence 2 for ms the oil phase. Sequence 3, 4 and 5 are added to the emulsion.

TABLE 3
Cellulite Ingredients for CELLUTONE ® Formulation

| SE- | | | |
|---|---|---|---|
| QUENCE | TRADENAME | INCI NAME | % W/W |
| 1 | Water | Water (Aqua) | 40-60 |
| 1 | Triethanola- | Triethanolamine | 0.30-3.00 |
| | mine 99% | | |
| 1 | Carbowax 400 | PEG-8 | 0.10-10.00 |
| 1 | Glycerin | Glycerin | 0.50-5.00 |
| 1 | Propylene Glycol | Propylene Glycol | 1.00-4.00 |
| 1 | Methylparaben | Methylparaben | 0.10-1.00 |
| 2 | Lipo GMS 450 | Glyceryl Stearate | 0.25-5.00 |
| 2 | Sweet Almond Oil | Sweet Almond | 0.60-6.00 |
| | | (Prunus Amygdalus | |
| | | Dulcis) Oil | |
| 2 | Ceraphyl 28 | Cetyl Lactate | 0.50-5.00 |
| 2 | Stearic Acid XXX | Stearic Acid | 0.50-5.00 |
| 2 | Paraffin | Paraffin | 0.50-5.00 |
| 2 | Sorbitan Stearate | Sorbitan Stearate | 0.50-5.00 |
| 2 | Propylparaben | Propylparaben | 0.10-5.00 |
| 2 | Dow Corning 3225C | Cyclomethicone | 0.10-10.00 |
| | | (and) Dimethicone | |
| | | Copotyol | |
| 2 | Whole Egg | Whole Egg | 0.10-10.00 |
| 3 | Grapefruit Oil | Grapefruit (Citrus) | 0.10-2.00 |
| | | Gandis) Oil | |
| 3 | Lavender Oil | Lavender (Lavendula | 0.50-5.00 |
| | | Angustifoli) Oil | |
| 3 | Geranium Oil | Geranium | 0.50-5.00 |
| | | Maculatum Oil | |
| 3 | Juniperberry Oil | Juniperus | 0.50-5.00 |
| | | Communis Oil | |
| 4 | Cumin Extract | Cumin (Cuminum | 0.50-5.00 |
| | | Cyminum) Extract | |
| 4 | Elderflower | Sambucus Nigra | 0.50-5.00 |
| | Extract | Extract | |
| 4 | Caraway Extract | Caraway (Carum | 0.50-5.00 |
| | | Carvi) Extract | |
| 4 | Sage Extract | Sage (Salvia | 0.50-5.00 |
| | | Officinalis) | |
| | | Extract | |
| 4 | Parsley Extract | Parsley (Carum | 0.50-5.00 |
| | | Petroselinum) | |
| | | Extract | |
| 4 | Cowslip Extract | Primula Veris | 0.50-5.00 |
| | | Extract | |
| 5 | Emmeressence | Phenoxyethanol | 0.10-3.00 |
| | 1160 Rose | | |

Below is a manufacturing process that is suitable for all three products which depicts a unique method of the addition of the water-soluble egg into the oil phase of the formulation at relatively high temperatures. The process allows the whole egg to be encapsulated by the oils and permits the formulation containing the encapsulated whole egg to penetrate into the skin and deliver the active ingredients. The egg within the encapsulated oil droplets is termed “Ovasomes®” and is formed prior to the emulsification process.

The “Ovasome Technology®” of the present invention is capable of carrying and delivering any and all types of ingredients, active and inactive, natural and synthetic, drugs, nutrients, or bioactives. The formulation of the present invention can also be modified to function as a cosmetic and/or pharmaceutical formulation depending on the additional ingredients added to this unique manufacturing process and depending on what ingredients may be desired to be delivered to a human or animal target. In one embodiment, the formulation of the present invention may be simply applied to the skin and the semi-permeable nature of the skin will allow entrance of the effective ingredient to be delivered by the “Eggosome” or “Ovasomes®” to the desired target. Since the “Eggosome” or “Ovasomes®” is capable of carrying various ingredients including active drugs, the formulation of the present invention may also have medicinal applications. The present invention may also include accelerated healing ingredients that may be applied to adhesive patches and bandages for commercial and hospital use.

### EXAMPLE 4

In Example 4, the shelf life of the skin care formulation of the present invention is tested. Five samples of Healfast™, five samples of Cellutone® and five samples of Ultra Soothe® were tested for shelf life. Stabilizing whole egg in skin care formulations and extending the shelf life of skin care products containing whole egg have been issues for chemist and chemical engineers who formulate skin care products.

Table 4 below shows the shelf life of the tested samples:

TABLE 4

| Sample: | Year #1: | Year #2: | Year #3: | Year #4 |
|---|---|---|---|---|
| Healfast #1 | OK | OK | OK | No |
| Healfast #2 | OK | OK | OK | No |
| Healfast #3 | OK | OK | OK | OK |
| Healfast #4 | OK | OK | No | No |
| Healfast #5 | OK | OK | OK | No |
| Cellutone #1 | OK | OK | No | No |
| Cellutone #2 | OK | OK | OK | No |
| Cellutone #3 | OK | OK | OK | No |
| Cellutone #4 | OK | OK | OK | No |
| Cellutone #5 | OK | OK | OK | No |
| Ultra Soothe #1 | OK | OK | OK | No |
| Ultra Soothe #2 | OK | OK | No | No |
| Ultra Soothe #3 | OK | OK | OK | No |
| Ultra Soothe #4 | OK | OK | OK | No |
| Ultra Soothe #5 | OK | OK | No | No |

Based on the shelf life testing, it has been established that the formulations of the present invention has a shelf life of at least one year. In fact, the testing indicates that the formulation has a shelf life of two years or more.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A cellulite formulation comprising whole egg, at least one herbal extract, at least one humectant substance and at least one aromatherapeutical substance, said formulation is used to reduce the visible appearance of cellulite, said formulation containing no caffeine, no aminophylline and no caffeine derivatives.

2. The formulation of claim 1 wherein said herbal extract is selected from a group consisting of cumin extract, elder berry extract, caraway extract, sage extract, parsley extract, cowslip extract, grapefruit seed extract and mixtures thereof.

3. The formulation of claim 1 wherein said herbal extract is selected from a group consisting of angelica extract, ashwaganda extract, astragalus extract, bilberry extract, black cohosh extract, blue cohosh extract, blessed thistle extract, burdock extract, California poppy extract, cactus extract, celandine extract, chamomile extract, chaparral extract, chaste tree extract, chickweed extract, cleavers extract, coltsfoot extract, corn silk extract, cramp bark extract, damiana extract, dandelion extract, devil's claw extract, dong quai extract, echinacea extract, sib ginseng extract, eyebright extract, false unicorn root extract, fenugreek extract, ginkgo extract, ginseng root extract, goldenrod extract, goldenseal extract, gotu kola extract, horsetail extract, hyssop extract, kava kava extract, lady's mantle extract, licorice root extract, milk thistle extract, motherwort extract, nettle extract, oats extract, Oregon grape root extract, osha root extract, passionflower extract, pau d'arco extract, plantain extract, red clover extract, red root extract, reishi mushroom extract, sarsaparilla extract, saw palmetto extract, St. John's wort extract, schisandra berry extract, shepherds purse extract, skullcap extract, slippery elm extract, usnea lichen extract, uva ursi extract, valerian extract, wild cherry extract, wild yam extract, wormwood extract, yellow dock extract, yucca root extract and mixtures thereof.

4. The formulation of claim 1 wherein said humectant substance is selected from a group consisting of glycerin, glycols, butylene glycol, propylene glycol, pentylene glycol and mixtures thereof.

5. The formulation of claim 1 wherein said aromatherapeutical substance comprises essential oils, said essential oils are selected from a group consisting of lavender oil, geranium oil, juniper berry oil, bay leaf oil, lemon oil, mandarin orange oil, orange oil and mixtures thereof

6. The formulation of claim 1 wherein said aromatherapeutical substance comprises essential oils, said essential oils are selected from a group consisting of absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetivewr oil, wintergreen oil, wild verbena oil, wormwood oil, yarrow oil, ylang ylang oil and mixtures thereof.

7. The formulation of claim 1 further comprising at least one emollient substance, said emollient substance is selected from a group consisting of sweet almond oil, Squalene, apricot oil, safflower oil, sunflower oil and mixtures thereof

8. The formulation of claim 1 wherein said whole egg is selected from a group consisting of poultry eggs, reptile eggs and mixtures thereof, said poultry eggs being selected from a group consisting of chicken eggs, duck eggs, emu eggs, geese eggs, guinea fowl eggs, ostrich eggs, partridge eggs, peafowl eggs, pheasant eggs, pigeon eggs, rhea eggs, swan eggs, turkey eggs and mixtures thereof.

9. The formulation of claim 1 wherein said formulation has a shelf life of at least one year.

10. The formulation of claim 1 wherein said whole egg comprises dehydrated whole egg.

11. The formulation of claim 8 wherein said dehydrated egg is hydrated during the manufacturing process of said formulation.

12. The formulation of claim 10 wherein said dehydrated whole egg comprises whole egg powder.

13. The formulation of claim 1 wherein said whole egg comprises hydrolyzed whole egg.

14. An application of use for treating cellulite, said application comprises whole egg, at least one essential oil, at least one humectant substance and at least one herbal extract, said essential oil is selected from a group consisting of lavender oil, geranium oil, juniper berry oil, bay leaf oil, lemon oil, mandarin orange oil, orange oil and mixtures thereof, said humectant substance is selected from a group consisting of glycerin, glycols, butylene glycol, propylene glycol, pentylene glycol and mixtures thereof and herbal extract is selected from a group consisting of cumin extract, elder berry extract, caraway extract, sage extract, parsley extract, cowslip extract, grapefruit seed extract and mixtures thereof.

15. The application of use of claim 14 further comprises at least one emollient substance, said emollient substance is selected from a group consisting of sweet almond oil, Squalene, apricot oil, safflower oil, sunflower oil and mixtures thereof.

16. The application of use of claim 14 wherein said application contains no caffeine and no aminophylline.

17. The application of use of claim 14 wherein said whole egg comprises whole egg powder.

18. The application of use of claim 17 wherein said whole egg powder is hydrated during the manufacturing process of said formulation.

19. The application of use of claim 14 wherein said herbal extract is selected from a group consisting of angelica extract, ashwaganda extract, astragalus extract, bilberry extract, black cohosh extract, blue cohosh extract, blessed thistle extract, burdock extract, California poppy extract, cactus extract, celandine extract, chamomile extract, chaparral extract, chaste tree extract, chickweed extract, cleavers extract, coltsfoot extract, corn silk extract, cramp bark extract, damiana extract, dandelion extract, devil's claw extract, dong quai extract, echinacea extract, sib ginseng extract, eyebright extract, false unicorn root extract, fenugreek extract, ginkgo extract, ginseng root extract, goldenrod extract, goldenseal extract, gotu kola extract, horsetail extract, hyssop extract, kava kava extract, lady's mantle extract, licorice root extract, milk thistle extract, motherwort extract, nettle extract, oats extract, Oregon grape root extract, osha root extract, passionflower extract, pau d'arco extract, plantain extract, red clover extract, red root extract, reishi mushroom extract, sarsaparilla extract, saw palmetto extract, St. John's wort extract, schisandra berry extract, shepherds purse extract, skullcap extract, slippery elm extract, usnea lichen extract, uva ursi extract, valerian extract, wild cherry extract, wild yam extract, wormwood extract, yellow dock extract, yucca root extract and mixtures thereof

20. The application of use of claim 14 wherein said essential oils are selected from a group consisting of absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsm oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalnatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetivewr oil, wintergreen oil, wild verbena oil, wormwood oil, yarrow oil, ylang ylang oil and mixtures thereof

21. A method of manufacturing a cellulite product, said method comprises admixing whole egg, at least one herbal extract, at least one humectant substance and at least one aromatherapeutical substance, said aromatherapeutical substance comprising essential oils, said formulation is used to reduce the visible appearance of cellulite, said formulation containing no caffeine, no aminophylline and no caffeine derivatives.

22. The method of claim 21 further comprises admixing aloe barbadensis leaf juice and at least one emollient.

23. The method of claim 21 wherein said cellulite product has a shelf life of at least one year.

24. The method of claim 21 wherein said whole egg comprises dehydrated whole egg, said dehydrated whole egg is whole egg powder.

25. The method of claim 21 wherein said whole egg powder is hydrated during the manufacturing method of said product.

26. The method of claim 21 wherein said herbal extract is selected from a group consisting of cumin extract, elder berry extract, caraway extract, sage extract, parsley extract, cowslip extract, grapefruit seed extract, angelica extract, ashwaganda extract, astragalus extract, bilberry extract, black cohosh extract, blue cohosh extract, blessed thistle extract, burdock extract, California poppy extract, cactus extract, celandine extract, chamomile extract, chaparral extract, chaste tree extract, chickweed extract, cleavers extract, coltsfoot extract, corn silk extract, cramp bark extract, damiana extract, dandelion extract, devil's claw extract, dong quai extract, echinacea extract, sib ginseng extract, eyebright extract, false unicorn root extract, fenugreek extract, ginkgo extract, ginseng root extract, goldenrod extract, goldenseal extract, gotu kola extract, horsetail extract, hyssop extract, kava kava extract, lady's mantle extract, licorice root extract, milk thistle extract, motherwort extract, nettle extract, oats extract, Oregon grape root extract, osha root extract, passionflower extract, pau d'arco extract, plantain extract, red clover extract, red root extract, reishi mushroom extract, sarsaparilla extract, saw palmetto extract, St. John's wort extract, schisandra berry extract, shepherds purse extract, skullcap extract, slippery elm extract, usnea lichen extract, uva ursi extract, valerian extract, wild cherry extract, wild yam extract, wormwood extract, yellow dock extract, yucca root extract and mixtures thereof

27. The method of claim 21 wherein said humectant substance is selected from a group consisting of glycerin, glycols, butylene glycol propylene glycol, pentylene glycol and mixtures thereof.

28. The method of claim 21 wherein said essential oils are selected from a group consisting of lavender oil, geranium oil, juniper berry oil, bay leaf oil, lemon oil, mandarin orange oil, orange oil, absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetivewr oil, wintergreen oil, wild verbena oil, wormwood oil, yarrow oil, ylang ylang oil and mixtures thereof.
